(19) United States
(12) Patent Application Publication
(10) Pub. No.: US 2009/0012392 A1
(43) Pub. Date: Jan. 8, 2009

(54) METHOD AND DEVICE FOR TISSUE CHARACTERIZATION

(76) Inventor: Dror Nir, Uccle (BE)

Correspondence Address: KNOBBE MARTENS OLSON & BEAR LLP, 2040 MAIN STREET, FOURTEENTH FLOOR, IRVINE, CA, 92614, US

(21) Appl. No.: 11/597,041

(22) PCT Filed: May 27, 2005
(86) PCT No.: PCT/IB2005/001506

§ 371 Date: Sep. 26, 2007

Related U.S. Application Data
(60) Provisional application No. 60/574,853, filed on May 27, 2004.

(51) Int. Cl. A61B 8/00

(52) U.S. Cl. 600/437

(57) ABSTRACT

Analytical methods which are complementary to ultrasonic imaging systems and relate to the medical classification of human tissue as healthy or unhealthy (e.g. malignant) are disclosed. Methods and systems for the detection of abnormal tissue, in particular the characterization of tissue morphology which typifies a cancer process, are disclosed. The methods and systems are particularly applicable to ovarian cancer.

### FIELD OF THE INVENTION

The present invention relates to analytical methods which are complementary to ultrasonic imaging systems and relates to the medical classification of human tissue as healthy or unhealthy (e.g. malignant). In particular the present invention relates to systems and method for the detection of abnormal tissue, in particular the characterization of tissue morphology which typifies cancer process. The invention is particularly adapted to cancer detection such as ovarian cancer.

### BACKGROUND OF THE INVENTION

Ultrasonic imaging systems are widely used in the medical diagnostic field for their ability to obtain the image of an object non invasively, i.e., by transmitting ultrasound to the object and processing its reflection. An ultrasonic imaging apparatus scans the interior of a subject to be imaged by an ultrasonic beam, receives an echo, generates image data and produces what is generally called a B-mode image based on the image data.

The ultrasound image, as seen on the apparatus screen is composed of grey levels which are the result of application of post processing mathematical procedures to the backscattered sound waves. The radiologist eye is trained to recognize tissue's qualities from the way his ultrasound system outputs the gray scale information as a video image onto the screen—mainly by examining the shape and size of the organ and parts of organ under examination.

In conventional 2D scanning the operator moves a transducer across an organ, whilst assessing separate video images (each representing a single ‘slice’ of tissue) and, simultaneously attempting to detect any deviations from what his memory and experience tell him is a reflection of normal pathology. Current virtual 3D technology uses special transducers that automatically sample a whole volume of tissue into many slices taken at regular intervals. These machines output a 3D image that can be further manipulated by the echographist.

The current status and reliability of imaging-based diagnosis is principally dependent on the interpretative ability of an individual practitioner who has to decide whether an image he sees is abnormal and furthermore is the abnormality is due to normal tissue proliferation, a benign neoplasm or a malignant process. The transformation of the ultrasound signal into video imaging implies distortion and loss of the immense amounts of information on tissues conveyed by the backscattered signal.

In oncology, ultrasound is mainly used for monitoring biopsy. Prior art screening methods based on echographic examination using standardized morphological criteria and measurement do not appear optimal. These techniques relay on image analysis techniques where quantification can only be applied to parameterization of characteristics as measured on the visual image. Hence, these prior art techniques are not using the information related to the intrinsic characteristics of the microscopic scatterers within a given tissue or region of tissue. US-imaging and serum tumour markers suffer from consistently high false negative “sensitivity” rates of ˜10% and false positive “specificity” rated of ˜25%. These figures appear very difficult to reduce.

Texture characterization of human body tissues has never been easy to perform. Quantitative methods used included fractal models, multichannel methods, analysis of first and second-order statistics of texture, Gaussian Markov random fields, mean scatterer spacing and multiresolution analysis. Invented algorithms are complicated and often inefficient.

Accordingly, it is an object of the present invention to overcome deficiencies in the prior art, such as indicated above. It is another object of the invention to detect the presence of disease in tissue on a quantitative basis. It is yet another object to provide an improved method of tissue characterization

### SUMMARY OF THE INVENTION

In a first aspect the present invention provides a method for tissue characterization for example for detecting abnormal tissue within a Region Of Interest (ROI) comprising the steps of:

a) obtaining RF data from a ROI within a tissue,
b) converting the RF data into Real Data,
c) converting the Real Data into Absolute Value Data, and
d) further processing the Absolute Value Data using one or more characterization algorithms in order to characterize said tissue into for example normal or abnormal tissue. The present invention permits differentiation between different pathologies.

In an embodiment the present invention provides a method for tissue characterization comprising the steps of:

a) obtaining RF data from ROI within a tissue,
b) converting the RF data into Real Data,
c) converting the Real Data into Absolute Value Data,
d) converting the Absolute Value Data to Grey Data, and
e) further processing the Grey Data using one or more characterization algorithms in order to characterize said tissue into normal or abnormal tissue.

Unless otherwise stated, the following terms used in this application, including the specification and claims, have the definitions given below.

Whenever used in the present invention the term “Region of interest (ROI)” refers to n dimensional space domain within the recorded tissue data, either the RF data or the Absolute Value Data or the Grey Data.

The term “RF data” refers to the backscattered Ultrasound (US) analog signals in the time domain.

The term “Real Data” refers to the RF data after digitization and Fast Fourier Transform (FFT). This is a matrix wherein each value is related to a specific spatial location, in the frequency domain, inside the recorded tissue. Each value is a real number represented by amplitude and phase and has a spatial position.

The term “Absolute Value Data” refers to an n dimensional matrix containing values which are the absolute values of the real matrix. Usually not less then 8 bit data per value are used to represent this data in the computer, preferably 16 or 32 bits are used. The values usually range between 0 to 5 volt (in a resolution determined by the number of bits allocated per value). The transition from Real Data to Absolute Value Data presents a new opportunity to analyze this data, which is “2^{nd} best” in quality compared to the Real Data, as it preserves the original resolution of the Real Data. The information that is lost in the Real Data to Absolute Value Data transition is that that was represented by the phase data. The phase data represents information of dynamic processes in the tissue (such as blood flow and elasticity) which are not utilized by the present invention.

The term “Grey Data” refers to the translation of the Absolute Value Data to gray levels. The grey levels are positive integers in the range 0 to MAX, 0 corresponding to totally black (no energy) and MAX corresponding to totally white. In the present invention a Grey Data is used, which was preferably created by an approximation to a linear translation between the absolute data and the gray data. Usually, 8 bit grey level data are used, in which case MAX=255, but it is possible, if available, to use also data with higher bit range (for example 16 bits or 32 bits). The linear translation has the advantage that it preserves best the inter relations between the different values as they exist in the Absolute Value Data.

The term “Characterization algorithms and thresholds” includes mathematical evaluation of the obtained Grey Data within the ROI.

The term “abnormal tissue” refers to tissue having a morphology different from normal tissue. For example abnormal tissue can be malignant tissue.

In a second aspect the present invention provides systems for tissue characterization as defined in the appended claims.

In a third aspect the present invention provides computer readable media containing computer-executable code tissue characterization as defined in the appended claims.

The present methods and systems enhance the non-invasive, ultrasound-based detection of cancers in solid organs, by performing accurate characterization of local tissue morphology. The present invention supplements ultrasound examination and helps corroborate the absence of malignant tissue arising from suspicious ultrasound findings. Thus, the present methods and systems have the potential to strengthen confidence in the watchful waiting treatment modality and helps reduce the need for invasive investigation when the presence of cancer is considered possible.

The present invention is applicable to the detection of several types of cancers non-limiting examples of which include ovarian, breast, prostate, liver, and kidney cancers.

### BRIEF DESCRIPTION OF THE FIGURES

Other objects and the nature and advantages of the present invention will be more apparent from the following detailed description of the invention taken in conjunction with the drawing, wherein:

FIG. 1 represents a block diagram of the data evolution according to an embodiment of the present method.

FIG. 2 represents a schematic block diagram describing the evolution chain of available ultrasound data according to an embodiment of the invention. The Grey Data volume matrix is the 4^{th} block before the rendering and 3D reconstruction algorithms are applied.

FIG. 3 represents a graph showing the influence of the machine Gain settings (Gain, TGC) on the mapping of ultrasound Real Data into Grey Data.

FIGS. 4a-4b represent graphs which illustrate differences in the regularity of backscattered signals between two sets of results that emerge from spectrum analysis of: (a) reflections from “normal” tissue, and (b) from “abnormal” tissue. X and Y axis are the space coordinates in the transformed matrix. The Z axis is the measure of energy: abnormal tissue reflection leads to lower ultrasound energy captured by probes (as compared to normal tissues). After transformation by characterization algorithms, the measures in the frequency domain lead to an apparent considerable increase (3 orders of magnitude) in the signature of phenomena (e.g., the disordering of tissue morphology) that led to lower backscattered ultrasound energy.

FIGS. 5a-5e represent graphs which illustrate the correlation applied to different characterization algorithms applied to data representing backscattering from malignant (left column: FIGS. 5a, 5c, 5d) tissue area and non-malignant (right column: FIGS. 5b, 5d, 5e) tissue area.

FIG. 6 represents video images of organs showing selected ROI and their corresponding grey level data according to a preferred embodiment of the invention.

FIG. 7 represents histograms wherein according to an embodiment of the invention, from Grey Data each algorithm computes a certain feature of the immediate surrounding tissue within the selected ROI. The results are collected in histogram plots shown therein (number of scores which have a certain value) and finally translated on a scale range.

FIG. 8 represents 3 graphs. Each graph illustrates the difference between benign (left) and malignant (right) sample. X axis=value of the Grey Data after transformation by one of the characterization algorithms. The y axis is the number of observations.

### DETAILED DESCRIPTION

In a first aspect, the present invention provides ultrasound-based technology for the characterization of tissue and more in particular for the detection of malignant tissue in solid organs. In particular the invention provides methods for tissue characterization which can be performed using signals from any 3D or 2D ultrasound (US) machine. The method according to the invention is devised for the analysis of non Real Data in the frequency domain. According to the present invention the specific characteristics of ultrasound backscattered data processed are not the Real Data but a derivative of the Real Data that is produced using specific procedures that preserves most of the required information needed for ensuring meaningful result upon analysis.

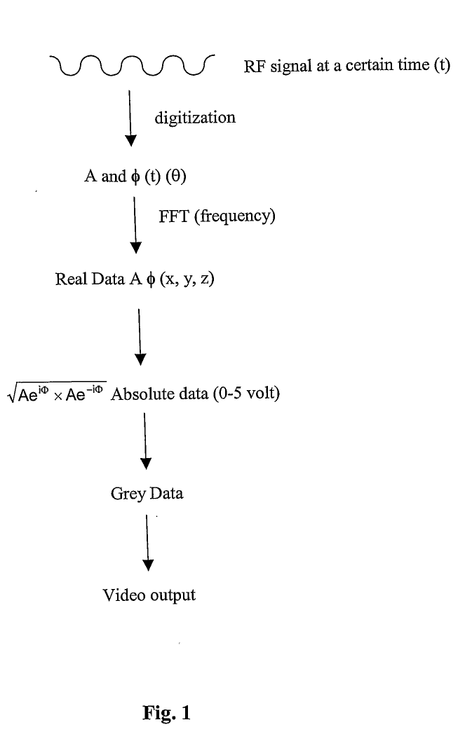

The method according to the invention is suitable for detecting abnormal tissue by specific data analysis. FIG. 1 illustrates an embodiment of the present method. The present method comprises, acquiring backscattered RF signals at a certain time (t). The ultrasounds reflected by tissues are measured in the time domain. The RF signals is then digitized to obtain a digitized signal phase φ and signal amplitude A at a time t and an angle node θ and said digitized signal is then submitted to a Fast Fourier Transform (FFT) to obtain Real Data wherein each value is a real number represented by amplitude A and phase φ and has a spatial position (x, y, z). The Real Data are generated from the RF signal by using, among other mathematical techniques power spectrum analysis.

The Real Data are then converted into Absolute Value Data according to the equation \sqrt{Ae^{iφ}×Ae^{-iφ}}. The Absolute Value Data can then be converted into Grey Data. Processing of the data can be performed either on the Absolute Value Data or on the Grey Data. In an embodiment of the present invention, the Grey Data is 8 bit Gray Data, i.e. containing numbers ranging between 0 to 255.

In a further embodiment of the present invention, the absolute data is converted into Grey Data by linear translation (or a good approximation thereof) of said Absolute Value Data. The linear translation has the advantage that it preserves the inter relations between the different values as they exist in the Absolute Value Data. A good approximation of a linear translation can be performed either by use of built in functions and settings of the ultrasound machine or by use of external functions. Non limiting example of a common linear transformation is Y=aX+b.

According to an embodiment of the present invention, assessing the level of tissue structure periodicity is done by analyzing either the Absolute Value Data or the Grey Data.

Table 1 represents an example of data manipulation according to the method of the present invention.

TABLE 1
Real Data
Amplitude

| | | | | | | | | | | |
|---|---|---|---|---|---|---|---|---|---|---|
| −158 | −292 | −38 | −57 | −379 | −158 | 173 | −232 | −195 | 882 | 791 |
| −138 | −220 | 32 | −177 | −493 | −133 | 1 | −442 | −208 | 622 | 255 |
| −93 | −121 | 102 | −300 | −530 | −108 | −120 | −555 | −185 | 308 | −224 |
| −22 | 7 | 137 | −408 | −478 | −73 | −209 | −573 | −152 | 3 | −551 |
| 42 | 125 | 131 | −453 | −333 | −38 | −282 | −508 | −123 | −236 | −684 |
| 56 | 167 | 77 | −416 | −120 | −6 | −361 | −399 | −111 | −364 | −632 |
| 3 | 119 | −32 | −296 | 97 | 23 | −440 | −281 | −111 | −404 | −452 |
| −121 | −16 | −163 | −131 | 256 | 36 | −491 | −163 | −114 | −379 | −251 |
| −293 | −202 | −270 | 28 | 310 | 26 | −492 | −53 | −97 | −323 | −118 |
| −448 | −373 | −322 | 134 | 233 | −8 | −416 | 47 | −61 | −278 | −107 |
| −528 | −463 | −274 | 127 | 59 | −68 | −255 | 128 | −1 | −269 | −178 |

Phase: The actual phase for computation purposes is the measured phase - n × 2

| | | | | | | | | | | |
|---|---|---|---|---|---|---|---|---|---|---|
| 8.08 | 8.37 | 8.64 | 8.73 | 8.84 | 8.76 | 8.29 | 8.36 | 8.64 | 8.42 | 8.54 |
| 8.06 | 8.38 | 8.63 | 8.69 | 8.80 | 8.69 | 8.23 | 8.37 | 8.65 | 8.48 | 8.61 |
| 8.04 | 8.43 | 8.65 | 8.65 | 8.73 | 8.60 | 8.15 | 8.38 | 8.65 | 8.50 | 8.65 |
| 8.02 | 8.49 | 8.73 | 8.71 | 8.71 | 8.54 | 8.06 | 8.38 | 8.63 | 8.50 | 8.65 |
| 7.99 | 8.52 | 8.77 | 8.78 | 8.72 | 8.47 | 8.00 | 8.36 | 8.60 | 8.45 | 8.61 |
| 7.95 | 8.54 | 8.80 | 8.79 | 8.69 | 8.38 | 7.94 | 8.33 | 8.56 | 8.43 | 8.63 |
| 7.91 | 8.54 | 8.80 | 8.76 | 8.62 | 8.27 | 7.86 | 8.30 | 8.53 | 8.47 | 8.67 |
| 7.86 | 8.55 | 8.77 | 8.70 | 8.52 | 8.12 | 7.80 | 8.25 | 8.49 | 8.47 | 8.67 |
| 7.79 | 8.56 | 8.78 | 8.62 | 8.40 | 7.97 | 7.75 | 8.20 | 8.44 | 8.44 | 8.64 |
| 7.71 | 8.55 | 8.78 | 8.57 | 8.24 | 7.79 | 7.71 | 8.12 | 8.36 | 8.37 | 8.58 |
| 7.61 | 8.54 | 8.78 | 8.51 | 8.02 | 7.65 | 7.70 | 8.04 | 8.28 | 8.30 | 8.53 |

Corresponding Absolute Value Data

| | | | | | | | | | | |
|---|---|---|---|---|---|---|---|---|---|---|
| 26.01 | 36.24 | 3.58 | 4.92 | 29.26 | 13.24 | 23.11 | 28.89 | 18.47 | 104.03 | 82.18 |
| 23.27 | 26.89 | 3.05 | 15.91 | 39.69 | 11.93 | 0.14 | 54.49 | 19.51 | 69.10 | 24.70 |
| 16.05 | 14.04 | 9.57 | 28.05 | 45.66 | 10.60 | 18.52 | 68.24 | 17.33 | 33.43 | 20.96 |
| 3.88 | 0.77 | 11.88 | 35.96 | 42.08 | 7.65 | 35.31 | 70.49 | 14.42 | 0.33 | 51.77 |
| 7.63 | 13.37 | 10.82 | 37.34 | 29.02 | 4.25 | 50.60 | 63.50 | 12.13 | 27.01 | 66.66 |
| 10.56 | 17.48 | 6.17 | 33.94 | 10.81 | 0.74 | 68.80 | 51.14 | 11.40 | 42.21 | 60.39 |
| 0.59 | 12.46 | 2.59 | 24.84 | 9.37 | 3.15 | 90.20 | 37.33 | 11.73 | 45.17 | 41.39 |
| 25.03 | 1.65 | 13.47 | 11.69 | 27.22 | 5.71 | 107.38 | 22.69 | 12.50 | 42.51 | 23.00 |
| 64.52 | 20.59 | 22.22 | 2.70 | 37.16 | 4.79 | 112.64 | 7.81 | 11.19 | 37.22 | 11.09 |
| 106.99 | 38.46 | 26.37 | 13.56 | 32.93 | 1.77 | 99.55 | 7.47 | 7.62 | 34.32 | 10.71 |
| 139.24 | 48.41 | 22.51 | 13.64 | 10.34 | 17.31 | 61.35 | 22.09 | 0.13 | 35.51 | 18.77 |

Corresponding Grey Data

| | | | | | | | | | | |
|---|---|---|---|---|---|---|---|---|---|---|
| 41 | 57 | 5 | 7 | 46 | 21 | 36 | 46 | 29 | 166 | 131 |
| 37 | 43 | 4 | 25 | 63 | 19 | 0 | 87 | 31 | 110 | 39 |
| 25 | 22 | 15 | 44 | 73 | 16 | 29 | 109 | 27 | 53 | 33 |
| 6 | 1 | 19 | 57 | 67 | 12 | 56 | 112 | 23 | 0 | 82 |
| 12 | 21 | 17 | 59 | 46 | 6 | 80 | 101 | 19 | 43 | 106 |
| 16 | 27 | 9 | 54 | 17 | 1 | 110 | 81 | 18 | 67 | 96 |
| 0 | 19 | 4 | 39 | 14 | 5 | 144 | 59 | 18 | 72 | 66 |
| 40 | 2 | 21 | 18 | 43 | 9 | 171 | 36 | 19 | 68 | 36 |
| 103 | 32 | 35 | 4 | 59 | 7 | 180 | 12 | 17 | 59 | 17 |
| 171 | 61 | 42 | 21 | 52 | 2 | 159 | 11 | 12 | 54 | 17 |
| 222 | 77 | 36 | 21 | 16 | 27 | 98 | 35 | 0 | 56 | 30 |

The diagram in FIG. 2 represents the evolution chain of available ultrasound data according to an embodiment of the present invention.

An example of the results of data manipulation is shown in FIG. 6 wherein video images of organs are represented showing selected ROIs and their corresponding Grey Data.

According to an embodiment of the present invention, the processing using characterization algorithm is applied to the Gray Data volume matrix (4^{th} block in FIG. 2) before rendering and 3D reconstruction algorithms are applied.

In an embodiment of the present invention, said characterization algorithm can be selected from the group comprising a Fourier analysis, a wavelet analysis and an entropy analysis. In a further embodiment the characterization algorithms may comprises one or more correlation algorithms.

The method according to the present invention is particularly suitable for detecting malignant tissue from acquired data, wherein the Absolute Value Data or Grey Data are processed with one or more characterization algorithms in order to determine whether the tissue is malignant based on a predetermined threshold. The present invention also provides a method for detecting malignant tissue within a ROI comprising the steps of: (a) obtaining RF data from a tissue comprising of a ROI, (b) converting the RF data into Real Data, (c) converting the Real Data into Absolute Value Data, and either (d) further processing the Absolute Value Data using one or more characterization algorithms and determining whether the tissue is malignant based on a predetermined threshold, or (d) converting the Absolute Value Data to Grey Data, and e) further processing the Grey Data using one or more characterization algorithms and determining whether the tissue is malignant based on a predetermined threshold.

Said predetermined threshold is determined by observing the characteristics histograms emerging from applying a characterization algorithm on a certain pathology in comparison to the histograms emerging from another pathology.

In an embodiment of the present invention, the processing of the Grey Data or Absolute Value Data can be either performed in real time or off line.

The method of the invention is applied to an area called ROI. The ROI is not necessarily a rectangle shape but could be any area of the whole matrix. According to a preferred embodiment the area is an n×m rectangular or polar pixel area, wherein n is equal to m or not. n and m are integers wherein n represents the number of rows of the matrix and m the number of columns of the matrix.

The novel method produces numerical discriminants between normal and abnormal tissue, and can further be applied to identify varying degrees of abnormality.

The objective of the present invention is detecting quantitatively the changes imposed on the backscattered waves by cancerous tissues. The present invention is aimed at measuring objectively characteristics of tissue morphology. Therefore, as long as it is applied to the Grey Data matrix it also comprises incorporation of: (i) special scanning methods, (ii) specific ultrasound transducer settings, (iii) use of specific translation functions from Real Data to Grey Data, to preserve optimal relation (close to linear) between the originally backscattered ultrasounds and their representing grey levels.

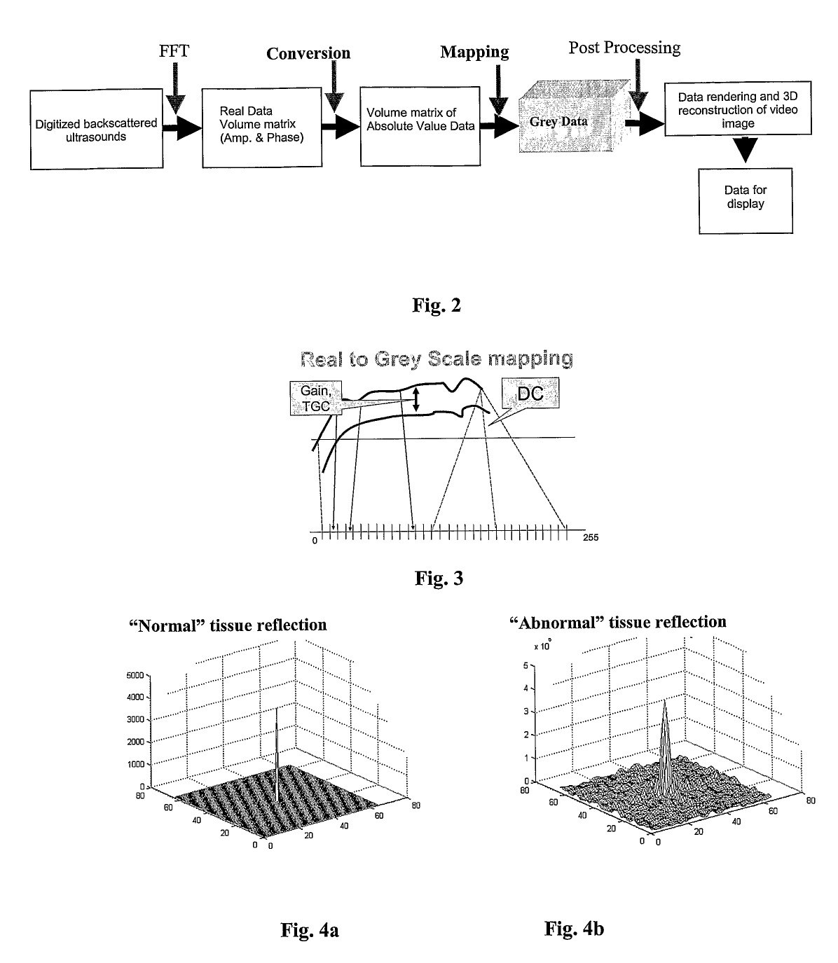

FIG. 3 represents a graph showing the influence of the machine Gain settings on the mapping of ultrasound Real Data into Grey Data. Gain parameter settings of the machine (Gain, TGC) are influencing the start position of the ZERO mapped value while the DC parameter settings influence the grey level range (by constraining the maximal grey level value) into which the whole signal is mapped.

The tissue characterization according to the present invention is based on the measurement of the divergences in the backscattered waves induced by abnormal tissues (e.g. FIG. 4).

The present invention is based on the use of mathematical methods or characterization algorithms comprising two main groups: characterization algorithms and correlation algorithms, that are best fitted to extract the changes induced in the backscattered waves by the presence of cancerous tissue.

In an embodiment, the characterization algorithms are selected from the group comprising a Fourier analysis, a wavelet analysis and an entropy analysis. The characterization algorithms are calibrated against different tissue pathologies. The present inventors have identified the characteristic results out of a matrix that represents non cancerous tissue and those that are characteristic of cancerous tissues. The meaning of “calibration” is the identification of those characteristics that are best separating the two pathological phenomena. This process is naturally organ dependant.

Suitable characterization algorithms for performing the present invention are sufficiently sensitive to changes in the backscattered energy induced by the alterations in the tissue morphology typical of the disease to be detected. Suitable characterization algorithms are described in U.S. Pat. No. 6,785,570 and PCT application WO 2004/000125 the subject matter of which is incorporated herein by reference.

In an embodiment, a Fourier transform F(y,ω)=∫I(x,y)e^{iwx} dx can be applied to each pixel. The energy of each Fourier transform can then be measured by evaluating the sum Σ|δFIδy| over the range of 1≦y≦28 and 34≦Ω≦64.

In another embodiment, a wavelet analysis can be performed using wavelet analysis software of the Matlab™ wavelet toolbox. A B-orthogonal filter can be used with a decomposition level equal to 1. The output of this software is four matrices known as the principle image coefficients (A), horizontal coefficients (H), vertical image coefficients (V) and the diagonal coefficients (D). Contour graph of the coefficients of A matrix can be obtained, and the maximum of each contour graph can be used as an index. Other indices maybe also used in accordance with the invention when using wavelet analysis such as the maximum coefficient in sum of the H, V and D coefficients matrices. Other filters may be used in accordance with the invention such as a Mexican hat filter, as are known in the art.

In yet another embodiment, an entropy analysis may be performed wherein for each pixel I(x,y), a parameter A(x,y) can be calculated by

$$A ( x , y ) = \frac{1}{n} \sum {\vert I ( x , y ) - I ( {x}^{′} , {y}^{′} ) \vert}^{2}$$

where the sum extends over all pixels (x′,y′) in the square neighboring the pixel (x,y), and n is the number of pixel (x, y). The entropy can then be calculated as the average of the A(x,y) over the entire square.

The set of correlation algorithms measures the amount of “similarity” or “dissimilarity” between results of analysis of the invention characterization algorithms, and it is first applied to Absolute Value Data or Grey Data belonging to samples of tissue (for example 20×20 pixel area) known as malignant or non-malignant. The tissue areas analyzed pathology is proven by standard pathology report. Further, the more similar the analysis results of a new data set to the analysis results from a known malignant sample (the higher the correlation probability on a 0 to 1 scale), the higher the probability that the current analyzed sample relates to a malignant tissue area. Each correlation algorithm results in a probability value (on a scale from 0 to 1, with 0 the least probable to be malignant and 1 the most probable to be malignant) which is further mapped onto a 0 to 7 scale (with 0 the least probable and 7 the most probable to be malignant tissue), so each correlation algorithm will result in a certain score. The final correlation score, of the currently analyzed 20×20 pixel area, is calculated by cumulating the scores of each correlation algorithm in part and a final score (probability) is issued. The present method is not limited to an area of 20×20 pixels.

In a non-limiting embodiment, a description of the details of the implementation of the present invention for the detection of ovarian cancer is given hereinafter.

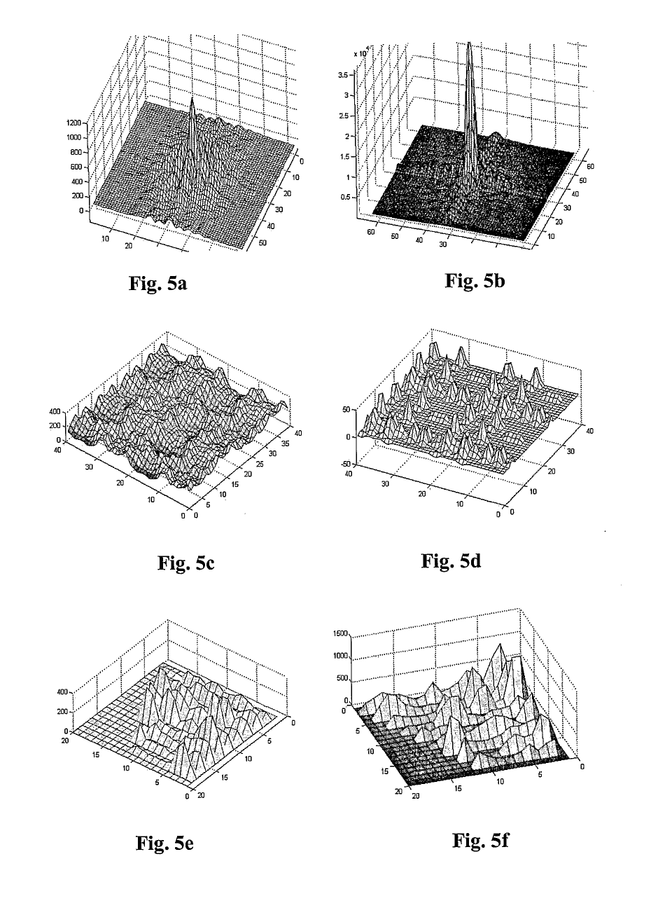

FIGS. 5a-5e show typical results of applying the characterization algorithms to malignant and non-malignant data samples. These results are used as input into the correlation algorithms applied subsequently. Application of each correlation algorithm results in a probability of similarity of the current sample to a malignant or non-malignant known sample. The probability is mapped to a scale of 0 to 7, with 0 meaning the least probable malignant and 7 the most probable malignant for “unknown to malignant proved” correlation, and 0 meaning the least probable non-malignant and 7 the most probable non-malignant for “unknown to non-malignant proved” correlation.

FIG. 8 also show typical results of applying characterization algorithms to malignant and non-malignant Grey Data samples. The results are plotted as histograms wherein in each histogram the difference between benign (left) and malignant (right) sample is shown.

The characterization algorithms chosen for the present invention can be applied simultaneously on successive 2D matrixes of Grey Data.

In an embodiment of the invention, in each slice, the characterization and correlation algorithms are applied to subgroups of 20×20 pixels of Grey Data with an overlap (currently 50%). The present invention permit high resolution, for one 20×20 pixel window may corresponds to a volume of 2×2 mm, having a depth of 0.15 to 0.40 mm.

The processing procedure for each 20×20 pixels area is composed of a set of successive groups of mathematical manipulations:
Each characterization algorithm generates a specific transformed matrix out of the original 20×20 pixels matrix.
From each transformed matrix, extraction and quantification of the statistical features that discriminate between the “normal” and “abnormal” characteristics of the tissues can be performed as described in U.S. Pat. No. 6,785,570 and PCT application WO 2004/000125.
Each correlation algorithm is applied on a matrix resulting from some of the characterization algorithms. The results of the correlation algorithms are the probabilities with which the analyzed samples are similar to results related to malignant or non-malignant tissue areas. The probabilities calculated are then mapped into a dimensionless normalized scale to produce a 8-point score ranging from 0 to 7, wherein “0” stands for most probable “non-malignant” and “7” stands for the most probable “malignant”.

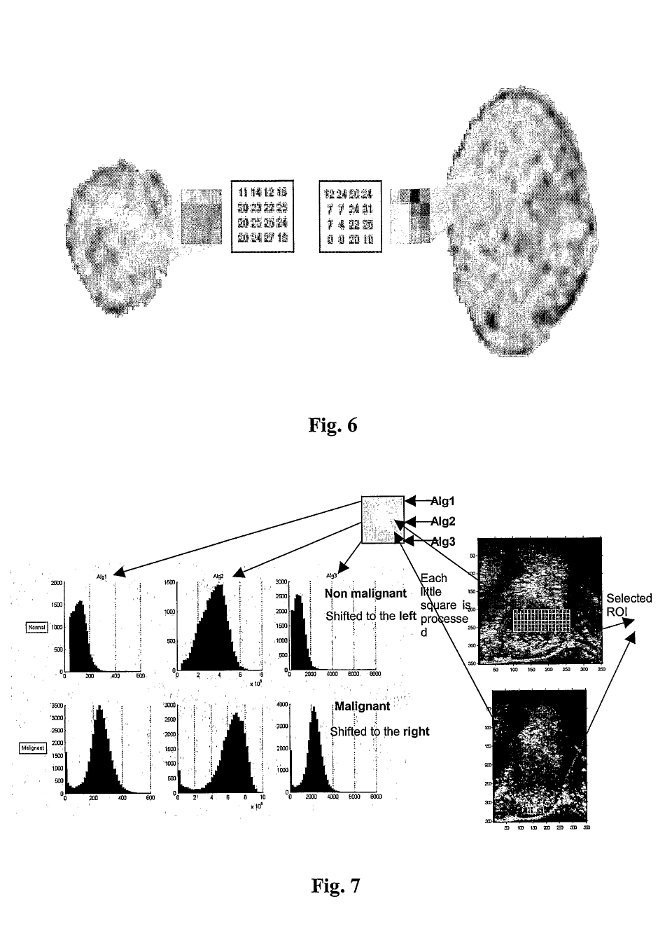

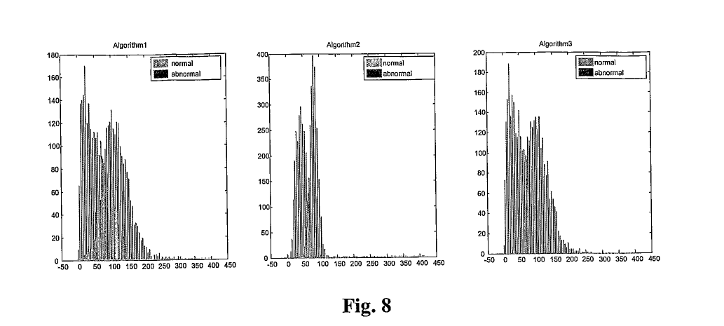

With respect to FIG. 7, from Grey Data each algorithm computes a certain feature of the immediate surrounding tissue (20×20 window) within the selected ROI. The results are collected in histogram plots (number of scores which have a certain value) and finally translated on a scale range.

The system report of the present method is in a form similar to that described in U.S. Pat. No. 6,785,570 and PCT application WO 2004/000125.

For example, the report can be in the form of a synthetic symbol (for example, a square), which is superimposed on a gray scale representation of the 2D organ slice. So, each 20×20 pixels area has its own score, independently from neighboring areas.

One more example is given where the different tissue types, as characterized by the invention, are expressed in different colors, and then these colors are overlaid in a semi-transparent layer on the image representation of the grey data, either in 3D or 2D view. For example, areas which have scores indicating cancerous tissue are added with a red highlight on the image so to visualize the detected tumor.

Data processing can be applied to Absolute Value Data or Grey Data stored on the ultrasound machine's hard drive by utilizing the ultrasound machine's internal processor or an independent computer who has access via the network to the ultrasound's machine hard drive. Alternatively, data processing can be applied to Absolute Value Data or Grey Data stored on personal computers, independent from the computer installed in the ultrasound machine, as long as the appropriate data files can be transferred from the ultrasound machine to the personal computer.

The present method can be used as a real-time analysis technique. So, the present method can be applied during an ultrasound examination session. Remote analysis is also possible, for instance, for echographists who would not have recourse to the present method on a regular basis.

The present detection method has the potential to be applied to a large variety of tissues and to identify abnormalities. The present detection method is sensitive and/or specific. The results of the present detection method can be compared to the results of a study of the pathology of the tissue or organ.

The present invention further encompasses a system for performing the method according to the present invention. In particular the present invention further provides a system for tissue characterization, comprising:
(a) a detector configured to detect and obtain RF data from a ROI within a tissue, and
(b) at least one processor configured to:
   (i) convert said RF data into Real Data,
   (ii) convert said Real Data into Absolute Value Data, and
   (iii) process the Absolute Value Data using one or more characterization algorithms configured to characterize said tissue into normal or abnormal tissue,
or
(b) at least two processors, a first processor configured to
   (i) convert said RF data into Real Data,
   (ii) convert said Real Data into Absolute Value Data, and
   a second processor configured to:
   (iii) process the Absolute Value Data using one or more characterization algorithms configured to characterize said tissue into normal or abnormal tissue.

The present invention also provides a system for tissue characterization comprising:
(a) a detector configured to detect and obtain RF data from a ROI within a tissue, and
(b) at least one first processor configured to
   (i1) convert said RF data into Real Data,
   (ii1) convert said Real Data into Absolute Value Data,
   (iii1) convert said Absolute Value Data to Grey Data, and
   (iii1) process said Grey Data using one or more characterization algorithms configured to characterize said tissue into normal or abnormal tissue,
or
(b) at least two processors, a first processor configured to
   (i1) convert said RF data into Real Data,
   (ii1) convert said Real Data into Absolute Value Data, and
   (iii1) convert said Absolute Value Data to Grey Data,
   a second processor configured to:
   (iii1) process said Grey Data using one or more characterization algorithms configured to characterize said tissue into normal or abnormal tissue.

In an embodiment, said processor is configured to convert said Absolute Value Data into Grey Data by linear translation of the Absolute Value Data.

The system according to the invention may further comprise a signal source configured to irradiate the tissue with Ultrasound (US) signals.

In an embodiment, the processor for use in the present system is configured to process the Absolute Value Data or the Grey Data using characterization algorithms which can be selected from the group comprising a Fourier analysis, a wavelet analysis and an entropy analysis. Said characterization algorithms may further comprise one or more correlation algorithms.

The present systems may be used to detect malignant tissue by configuring the processor to process the data with one or more characterization algorithms in order to determine whether the tissue is malignant based on a predetermined threshold.

The present invention also provides a computer readable medium comprising computer-executable code for tissue characterization which is characterized by performing the functions of:
a) obtaining RF data from a ROI within a tissue,
b) converting the RF data into Real Data,
c) converting the Real Data into Absolute Value Data,
d) further processing the Absolute Value Data using one or more characterization algorithms in order to characterize said tissue into normal or abnormal tissue.

The present invention also provides a computer readable medium comprising computer-executable code for tissue characterization which is characterized by performing the functions of:
a) obtaining RF data from a ROI within a tissue,
b) converting the RF data into Real Data,
c) converting the Real Data into Absolute Value Data,
d) converting the Absolute Value Data to Grey Data, and
e) further processing the Grey Data using one or more characterization algorithms in order to characterize said tissue into normal or abnormal tissue.

According to an embodiment of the present invention, the computer-executable code converts the Absolute Value Data into Grey Data by linear translation of said Absolute Value Data. In an embodiment said Grey Data is 8 bit Grey Data.

The computer-executable code processes the Absolute Value Data or the Grey Data using characterization algorithms selected from the group comprising a Fourier analysis, a wavelet analysis and an entropy analysis. In an embodiment the characterization algorithms may comprise one or more correlation algorithm.

The present invention also encompasses a computer readable medium comprising computer-executable code for detecting malignant tissue within a ROI, which is characterized by performing the functions of:
a) obtaining RF data from a ROI within a tissue,
b) converting the RF data into Real Data,
c) converting the Real Data into Absolute Value Data,
d) further processing the Absolute Value Data using one or more characterization algorithms in order to determine whether the tissue is malignant based on a predetermined threshold.

In an embodiment the computer readable medium comprises computer-executable code for detecting malignant tissue within a ROI, which is characterized by performing the functions of:
a) obtaining RF data from a tissue comprising of a ROI,
b) converting the RF data into Real Data,
c) converting the Real Data into Absolute Value Data,
d) converting the Absolute Value Data to Grey Data, and
e) further processing the Grey Data using one or more characterization algorithms in order to determine whether the tissue is malignant based on a predetermined threshold.

While the details of the present invention have been described with specific reference to a preferred embodiment and in the context of specific characterization of ovarian tissue, it is apparent that variations and applications may be made without departing from the spirit and scope of the inventive concept as defined by the appended claims.

1. A method for tissue characterization comprising the steps of:
a) obtaining RF data from a region of interest within a tissue,
b) converting the RF data into Real Data,
c) converting the Real Data into Absolute Value Data, and
d) further processing the Absolute Value Data using one or more characterization algorithms in order to characterize said tissue into normal or abnormal tissue.

2. A method for tissue characterization comprising the steps of:
a) obtaining RF data from a region of interest within a tissue,
b) converting the RF data into Real Data,
c) converting the Real Data into Absolute Value Data,
d) converting the Absolute Value Data to Grey Data, and
e) further processing the Grey Data using one or more characterization algorithms in order to characterize said tissue into normal or abnormal tissue.

3. The method according to claim 2 wherein step (d) comprises performing a linear translation of the absolute data to obtain said Grey Data.

4. The method according to claim 2, wherein said Grey Data is 8 bits Grey Data.

5. The method according to claim 1 or 2, wherein said characterization algorithm is selected from the group consisting of a Fourier analysis, a wavelet analysis and an entropy analysis.

6. The method according to claim 1 or 2, wherein said characterization algorithms comprise one or more correlation algorithm.

7. The method according to claim 1 or 2, for detecting malignant tissue, wherein said Absolute Value Data or Grey Data are processed with one or more characterization algorithms in order to determine whether the tissue is malignant based on a predetermined threshold.

8. The method according to claim 1 or 2, wherein said processing step is performed in real time or off line.

9. A System for tissue characterization, comprising:
(a) a detector configured to detect and obtain RF data from a region of interest within a tissue, and
(b) at least one processor configured to:
(i) convert said RF data into Real Data,
(ii) convert said Real Data into Absolute Value Data, and
(iii) process the Absolute Value Data using one or more characterization algorithms configured to characterize said tissue into normal or abnormal tissue,
or
(b) at least two processors, a first processor configured to:
(i) convert said RF data into Real Data,
(ii) convert said Real Data into Absolute Value Data, and
a second processor configured to:
(iii) process the Absolute Value Data using one or more characterization algorithms configured to characterize said tissue into normal or abnormal tissue.

10. A System for tissue characterization, comprising:
(a) a detector configured to detect and obtain RF data from a region of interest within a tissue, and
(b) at least one first processor configured to
(i1) convert said RF data into Real Data,
(ii1) convert said Real Data into Absolute Value Data,
(iii1) convert said Absolute Value Data to Grey Data, and
(iii1) process said Grey Data using one or more characterization algorithms configured to
characterize said tissue into normal or abnormal tissue,
or
(b) at least two processors, a first processor configured to:
(i1) convert said RF data into Real Data,
(ii1) convert said Real Data into Absolute Value Data, and
(iii1) convert said Absolute Value Data to Grey Data,
a second processor configured to:
(iii1) process said Grey Data using one or more characterization algorithms configured to characterize said tissue into normal or abnormal tissue.

11. The system according to claim 10, wherein said processor is configured to convert said Absolute Value Data into Grey Data by linear translation of the Absolute Value Data.

12. The system according to claim 9 or 10, further comprising a signal source configured to irradiate the tissue with Ultrasound (US) signals.

13. The system according to claim 9 or 10, wherein the characterization algorithm is selected from the group consisting of a Fourier analysis, a wavelet analysis and an entropy analysis.

14. The system according to claim 9 or 10, wherein said characterization algorithms comprise one or more correlation algorithms.

15. A computer readable medium comprising computer-executable code for tissue characterization which is characterized by performing the functions of:
a) obtaining RF data from a region of interest within a tissue,
b) converting the RF data into Real Data,
c) converting the Real Data into Absolute Value Data, and
d) further processing the Absolute Value Data using one or more characterization algorithms in order to characterize said tissue into normal or abnormal tissue.

16. A computer readable medium comprising computer-executable code for tissue characterization which is characterized by performing the functions of:
a) obtaining RF data from a region of interest within a tissue,
b) converting the RF data into Real Data,
c) converting the Real Data into Absolute Value Data,
d) converting the Absolute Value Data to Grey Data, and
e) further processing the Grey Data using one or more characterization algorithms in order to characterize said tissue into normal or abnormal tissue.

17. The computer readable medium according to claim 16, wherein the computer-executable code converts the Absolute Value Data into Grey Data by linear translation of said Absolute Value Data.

18. The computer readable medium according to claim 15 or 16, wherein the characterization algorithm is selected from the group comprising a Fourier analysis, a wavelet analysis and an entropy analysis.

19. The computer readable medium according to claim 15 or 16, wherein said characterization algorithms comprise one or more correlation algorithm.
